(19) United States
(12) Patent Application Publication
(10) Pub. No.: US 2015/0132827 A1
(43) Pub. Date: May 14, 2015

(54) Emulsifier Centrifuge Tube Fermenter

(71) Applicant: MicroBioPlastic, L.P., Carlsbad, CA (US)

(72) Inventor: Andre Simoes, Vista, CA (US)

(21) Appl. No.: 14/533,517

(22) Filed: Nov. 5, 2014

Related U.S. Application Data
(60) Provisional application No. 61/903,936, filed on Nov. 13, 2013.

(51) Int. Cl. C12M 1/12; C12M 1/00; C12N 1/20

(52) U.S. Cl. CPC C12M 23/06; C12N 1/20; C12M 23/38; C12M 23/34
USPC 435/252.1; 435/304.2

(57) ABSTRACT

A centrifuge tube fermenter for culturing cells in a nutrient medium wherein the centrifuge tube fermenter is comprised of a top end, a bottom end, a vertical cylindrical outer wall positioned between the top end and the bottom end, and an inner wall positioned inside the outer wall, the bottom end further comprised of a plurality of baffles which protrude from inner wall toward a theoretical center of the vertical cylindrical outer wall terminating in a conical tip.

### RELATED APPLICATIONS

This application is a non-provisional patent application claiming the benefit of priority from U.S. Provisional Pat. App. Ser. No. 61/903,936 filed Nov. 13, 2013, the entire contents of which are incorporated by reference herein.

### FIELD OF THE INVENTION

The present invention pertains generally to centrifuge tubes. More particularly, the present invention pertains to emulsifier centrifuge tube fermenters with side and bottom baffles.

### BACKGROUND OF THE ART

Centrifuge tubes have numerous applications in different fields of biotechnology. In general, centrifuge tubes are small tubes which are typically tapered, conical or rounded and closed at one end. Such tubes are capable of holding 0.4 to 2.0 ml samples, often liquids, and are designed to withstand forces in excess of 10,000 times their own weight (10,000×g) during centrifugation. The tubes are used widely in laboratories as vessels for holding radioisotope chemicals, for storing biochemical, for performing biochemical reactions, and for handling contaminant free samples.

As is often the case in biotechnology research, performing biochemical reactions requires the rapid increase in volume of biological starting materials. Rapid growth of host organisms such as E. colt, S. cerevisiea, and Pichia in small vessels is limited because of poor nutrient and gas exchange during growth. For example, recovery after Plasmid mediated transformation of frozen competent E. coli is improved when cells are placed in larger tubes. This growth rate enhancement can increase the transformation efficiency by a factor of 25 fold for DH10B cells.

One alternative vessel for culturing cells is the fermentation flask, such as that disclosed by Ellis and Harlan in US. Pat. Pub. No. US2005/277191. The bottom of the flask has six equally spaced baffles extending inward from the rounded corners of the flask and upward toward a large diameter opening of the flask centered on the longitudinal axis. The baffles have a triangular cross-sectional shape with an included angle of about 28-40° and a height measured parallel to the longitudinal axis of about 15-25% of the usable flask volume. Although available, increasing the size of the culture vessel is not always a viable option or may not be the desired approach when starting material volumes are extremely small.

Examples of centrifuge tubes are found in the following prior art:

U.S. Pat. Pub. No. US2009/148941 by Florez et al. discloses a cylindrical cell culture tube with a cap having a septum and gas exchange membranes. In addition, the tube may optionally be customized to incorporate molded turbulence promoting baffles on the bottom sidewall such that, when combined with agitation on a rotational incubator shaker of appropriate amplitude, produces mixing sufficient to mimic that of large scale systems.

U.S. Pat. Pub. No. US2009/311775 by Kocourek et al. discloses a disposable bioreactor for culturing cells wherein the bioreactor is equipped with at least one baffle adjacent to the internal wall of the container forming a web, the web being arranged running vertically from the bottom upward, preferably in the shape of a helix.

In light of the above, it is an object of the present invention to provide the desired features described herein as well as additional advantages such as improving and expediting the living cell production growth rate by over 200%/unit volume.

### SUMMARY OF THE INVENTION

The present invention is directed to solving the deficiency in the art pertaining to the availability of centrifuge tube fermenters. It is one object of the present invention to provide an emulsifier centrifuge tube fermenter with side and bottom baffles.

It is another object of the present invention to provide a centrifuge tube fermenter with side and bottom baffles capable of improving and expediting the living cell production growth rate by over 200%/unit volume.

It is another object of the present invention to provide a centrifuge tube fermenter with side and bottom baffles wherein during agitation the baffles cause a greater percentage of culture medium and/or organism surface area to be exposed to oxygen. In a preferred embodiment, the increased exposure of the culture medium and/or organism surface area to oxygen results in increased aeration, growth and subsequent separation of organisms from culture medium.

It is another object of the present invention to provide a centrifuge tube fermenter with side and bottom baffles where the tube has an increased shelf-life and usage life during transport, storage and sample processing.

It is yet another object of the present invention to provide a method for improving cell culturing, the method comprising:

providing a centrifuge tube fermenter with side and bottom baffles having a screw on cap or snap cap;

placing cell culture media and one or more inocula cells inside the centrifuge tube fermenter;

allowing one or more cells to divide in the media to produce progeny cells;

centrifuging the centrifuge fermenter tube; and

removing the cell culture media by aspiration thereby leaving the cultured cell pellet for further use.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 illustrates a longitudinal cross section view of the centrifuge tube fermenter.

FIG. 2 illustrates a magnified view of the encircled bottom of the centrifuge tube fermenter of FIG. 1.

FIG. 3 illustrates a top view of the centrifuge tube fermenter of FIG. 1.

FIG. 4 illustrates the results of a cell growth comparison study performed using the centrifuge tube fermenter and standard 15 ml cell culture tube.

### DETAILED DESCRIPTION OF THE INVENTION

A centrifuge tube fermenter 1 for culturing cells in a nutrient medium is shown in FIG. 1. Centrifuge tube fermenter 7 is comprised of a top end 8 and a bottom end 7, the top end 8 having threads 3 for a screw-on or snap cap (not shown) and the bottom end 7 terminating in a conical tip 4. Centrifuge tube fermenter 7 is further comprised of a vertical cylindrical wall 5 positioned between top end 8 and bottom end 7. Vertical cylindrical wall 5 is further comprised of an inner wall 6. Bottom end 7 is further comprised of a plurality of baffles 1-1 which protrude from inner wall 6 toward the theoretical center 2 of the vertical cylindrical wall 5.

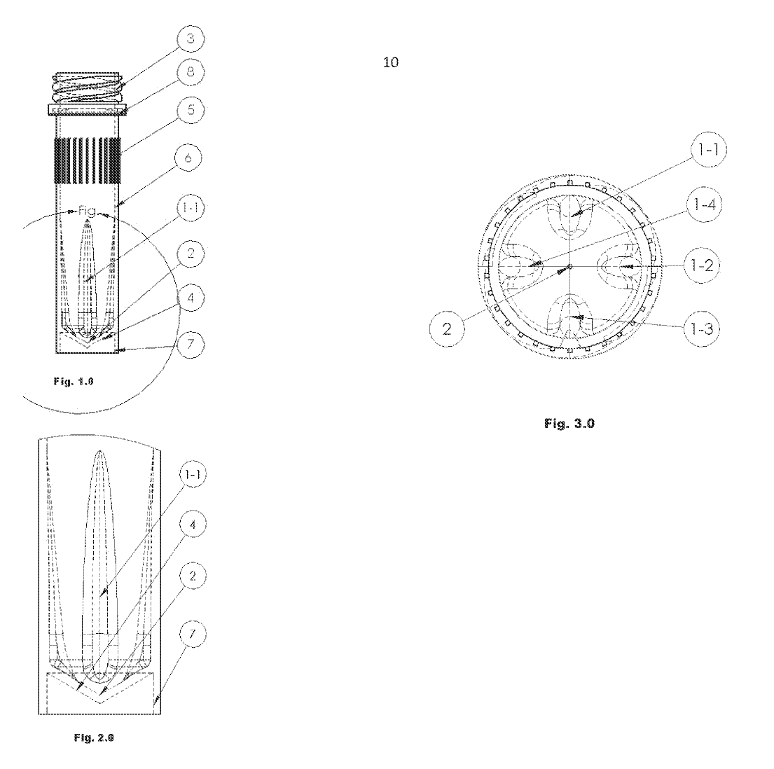

With reference to FIG. 2, a magnified view of the bottom end 7 of the centrifuge tube fermenter 1 shown in FIG. 1 is illustrated. The conical tip 4 is visible beneath the plurality of baffles 1-1 as is the theoretical center 2 of the vertical cylindrical wall 5.

With reference to FIG. 3, a top view of the centrifuge tube fermenter of FIG. 1 is illustrated. The position of the plurality of baffles (1-1, 1-2, 1-3, 1-4) is shown in relation to the theoretical center 2 of the vertical cylindrical wall (not shown). A plurality of baffles equals four baffles for the 2 ml centrifuge tube fermenter as shown. The number of baffles may be increased or decreased according to the volume of the centrifuge tube and the amount of increased aeration desired where the number of baffles will be at least one.

### EXAMPLE 1

Small-scale preparation of plasmid DNA was performed in the centrifuge tube fermenter of the present invention and a standard 15 ml culture tube in order to compare cell growth.

### Methods

A single bacterial colony was transferred into 0.5 ml of amp LB medium, containing 50 μg/ml ampicillin in each of a loosely capped 2.0 ml centrifuge tube fermenter and standard 15 ml culture tube. Cell cultures were incubated overnight at 37° C. with vigorous shaking. Culture tubes were centrifuged at 12,000 g for 30 seconds at 4° C. The supernatant was removed by aspiration and the cells in the bacterial pellet were counted and compared.

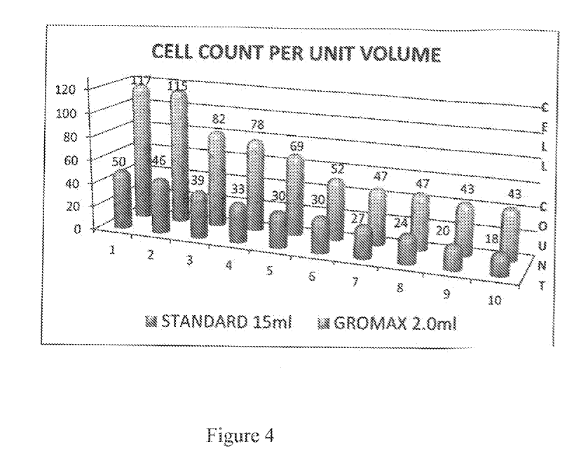

FIG. 4 shows the results of the cell growth comparison study. Cell counts of cultures grown in standard 15 ml cell culture tubes are shown in the front row while cell counts of cultures grown in the centrifuge tube fermenter of the present invention are shown in the back row. The cell count in the centrifuge tube fermenter increased by over 200% compared to the cell count in standard 15 ml tubes. These results confirm that the design of the centrifuge tube fermenter drastically increases bacterial culture growth per unit volume.

One embodiment of the present invention provides a centrifuge tube fermenter for culturing cells in a nutrient medium, the centrifuge tube fermenter comprised of:

a top end

a bottom end;

a vertical cylindrical outer wall positioned between the top end and the bottom end; and

an inner wall positioned inside the outer wall,

wherein the bottom end is further comprised of a plurality of baffles which protrude from inner wall toward a theoretical center of the vertical cylindrical outer wall. In a preferred embodiment the top end is further comprises of threads for a screw-on lid and the bottom end further terminated in a conical tip.

In another embodiment of the present invention, the centrifuge tube fermenter can be fabricated from any appropriate material, including but not limited to, polypropylene/polysulfone, polyethersulfone, ABS/polycarbonate, a polyacrylic plastic tube container and/or the like. In a preferred embodiment, the centrifuge tube fermentor is injection molded from virgin material polyolefin, such as high density polypropylene or polyethylene. In a most preferred embodiment, the centrifuge tube fermenter is injection molded from ultra-clear medical grade polypropylene.

In yet another embodiment of the present invention, the centrifuge tube fermenter may be fabricated in a variety of volumes ranging from 0.6 ml to 50 ml. In a preferred embodiment, the centrifuge tubes are micro-centrifuge tubes with a volume ranging from 250 μl to 2 ml.

It will be appreciated that details of the foregoing embodiments, given for purposes of illustration, are not to be construed as limiting the scope of the invention. Although several embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is further defined in the converted utility application and appended claims. Further, it is recognized that many embodiments may be conceived that do not achieve all the advantages of some embodiments, particularly preferred embodiments, yet the absence of a particular advantage shall not be construed to necessarily mean that such an embodiment is outside the scope of the present invention.

What is claimed is:

1. A centrifuge tube fermenter for culturing cells in a nutrient medium, the centrifuge tube fermenter comprised of:
a top end
a bottom end;
a vertical cylindrical outer wall positioned between the top end and the bottom end; and
an inner wall positioned inside the outer wall,
wherein the bottom end is further comprised of a plurality of baffles which protrude from inner wall toward a theoretical center of the vertical cylindrical outer wall terminating in a conical tip.

2. The centrifuge tube fermenter of claim 1, wherein the centrifuge tube fermenter is fabricated from an appropriate material selected from the group consisting of polypropylene/polysulfone, polyethersulfone, ABS/polycarbonate, poluolefin, polyethylene and polyacrylic plastic.

3. The centrifuge tube fermenter of claim 2, wherein the centrifuge tube fermenter is fabricated from polyolefin.

4. The centrifuge tube fermenter of claim 2, wherein the centrifuge tube is fabricated from polypropylene.

5. The centrifuge tube fermenter of claim 2, wherein the centrifuge tube fermenter is fabricated by injection molding.

6. The centrifuge tube fermenter of claim 1, wherein the centrifuge tube fermenter is fabricated with a total volume selected from the range of about 250 μl to about 50 ml.

7. The centrifuge tube fermenter of claim 6, wherein the centrifuge tube fermenter is fabricated with a total volume selected from the range of about 250 μl to 2 ml.

8. The centrifuge tube fermenter of claim 6, wherein the centrifuge tube fermenter is fabricated with a total volume selected from the range of about 0.6 ml to about 50 ml.

9. The centrifuge tube fermenter of claim 8, wherein the centrifuge tube fermenter is fabricated with a total volume of about 1-2 ml.

10. A centrifuge tube fermenter for culturing cells in a nutrient medium, the centrifuge tube fermenter comprised of:
a top end having a screw on cap or snap cap;
a bottom end;
a vertical cylindrical outer wall positioned between the top end and the bottom end; and
an inner wall positioned inside the outer wall,
wherein the bottom end is further comprised of a plurality of baffles which protrude from inner wall toward a theoretical center of the vertical cylindrical outer wall terminating in a conical tip.

11. The centrifuge tube fermenter of claim 10, wherein the centrifuge tube fermenter is fabricated from an appropriate material selected from the group consisting of polypropylene/polysulfone, polyethersulfone, ABS/polycarbonate, poluolefin, polyethylene and polyacrylic plastic.

12. The centrifuge tube fermenter of claim 11, wherein the centrifuge tube fermenter is fabricated from polyolefin.

13. The centrifuge tube fermenter of claim 11, wherein the centrifuge tube is fabricated from polypropylene.

14. The centrifuge tube fermenter of claim 11, wherein the centrifuge tube fermenter is fabricated by injection molding.

15. The centrifuge tube fermenter of claim 10, wherein the centrifuge tube fermenter is fabricated with a total volume selected from the range of about 250 μl to about 50 ml.

16. The centrifuge tube fermenter of claim 15, wherein the centrifuge tube fermenter is fabricated with a total volume selected from the range of about 250 μl to 2 ml.

17. The centrifuge tube fermenter of claim 15, wherein the centrifuge tube fermenter is fabricated with a total volume selected from the range of about 0.6 ml to about 50 ml.

18. The centrifuge tube fermenter of claim 17, wherein the centrifuge tube fermenter is fabricated with a total volume of about 1-2 ml.

19. A method for improving cell culturing, the method comprising:
providing a centrifuge tube fermenter with side and bottom baffles terminating in a conical tip having a screw on cap or snap cap;
placing cell culture media and one or more inocula cells inside the centrifuge tube fermenter;
allowing one or more cells to divide in the media to produce progeny cells;
centrifuging the centrifuge fermenter tube; and
removing the cell culture media by aspiration thereby leaving the cultured cell pellet for further use.

20. A method for improving cell culturing, the method comprising:
providing a centrifuge tube fermenter with side and bottom baffles terminating in a conical tip having a screw on cap or snap cap;
placing cell culture media and one or more inocula cells inside the centrifuge tube fermenter;
allowing one or more cells to divide in the media to produce progeny cells;
centrifuging the centrifuge fermenter tube; and
removing the cell culture media by aspiration thereby leaving the cultured cell pellet for further use.
